(19) United States
(12) Patent Application Publication
(10) Pub. No.: US 2011/0230424 A1
(43) Pub. Date: Sep. 22, 2011

(54) OPHTHALMIC SOLUTIONS, INCLUDING CONTACT LENS CARE AND EYE DROPS COMPRISING CARNOSINE, PREFERABLY IN COMBINATION WITH DEXPANTHENOL AND/OR HYALURONIC ACID

(76) Inventor: Louis Johan Wagenaar, Leiden (NL)

(21) Appl. No.: 13/129,709

(22) PCT Filed: Nov. 17, 2009
(86) PCT No.: PCT/NL2009/000220

§ 371 Date: May 17, 2011

(30) Foreign Application Priority Data
Nov. 17, 2008 (NL) 2002214

(51) Int. Cl. A61K 38/05; A61P 27/02

(52) U.S. Cl. 514/20.8

(57) ABSTRACT

The current invention relates to a contact lens care solution comprising a compound suitable for treating the eye, or the contact lens, use of such compound in such contact lens care solutions, and methods for introducing such compound in the eye of a person wearing contact lenses. With the current invention damage to the eye, in particular the cornea, in particular damage to the eye, in particular the cornea, of a person wearing a contact lens, or as a consequence of wearing a contact lens is prevented or reduced./esp.

### STATE OF THE ART

Contact lenses provide a means for vision correction for many consumers, improved convenience and appearance in comparison to spectacles probably being the most important advantages. However, contact lenses require regular care in order to ensure comfort and avoid ocular infections. Proper care of contact lenses typically requires to periodically clean, disinfect, and/or rinse the contact lenses. Cleaning relates to removal of dirt, lipids, proteins or other matter (biofilm) which has become affixed to a contact lens. Disinfecting relates to inactivating harmful bacteria or fungi and rinsing usually relates to removing debris from the contact lens before it is placed in the eye.

Disinfecting, cleaning and/or rinsing of lenses is performed by immersing a contact lens in a contact lens care solution like single- or multiple-purpose contact lens care solutions known in the art. Contact lenses are often left in such contact lens care solution for at least several hours. After treatment, the contact lenses are ready for wear.

However, experience shows that consumers have difficulties or are not always careful enough in regularly following the instructions of cleaning and caring for the contact lenses or are not taking enough time for (daily) cleaning and caring. Consequently this might lead to increased irritation of the eye or even damage to the eye, in particular the cornea, as a direct consequence of wearing contact lenses.

Treatment of damage, including for example starting lesions, in an early stage—as soon as possible in order to prevent further damage,—is an urging point. In fact preferably damage to the eye, in particular the cornea, or irritation of the eye, in particular the cornea, should be prevented from the start, i.e. before it occurs and in any case before it is manifested and has become a problem to the wearer of contact lenses.

In addition, temporary changes of the surface of the eye, for example swelling, for example as a consequence of allergens might lead to the circumstance that a contact lens, albeit temporarily, does not perfectly fit to the eye, which consequently might lead to (further) damage to the surface of the cornea.

This makes for contact lens wearers at least, eye drops for example less suitable, as a measure to prevent damage to the eye, in particular the cornea, for example damage due to wearing contact lenses. Such drops are generally only applied by a user once an eye-problem has been noticed, i.e. at the moment irritation and damage to the eye, in particular the cornea, have already occurred. In fact in many cases a subject will abstain from wearing contact lenses once an eye-problem is evident. Indeed only very few consumers will administer eye drops for prolonged periods of time only as a precautionary measure, or only to prevent possible damage to the eye, in particular to the cornea, in particular, damage to the eye, in particular the cornea, as the consequence of wearing contact lenses. However, this obviously does not exclude eye drops as a measure to prevent damage to the eye, in particular the cornea.

It is however important to constantly nourish the cornea/eye surface in order to maintain a sufficient health status. In particular, such nourishment is important when a subject is wearing contact lenses, which in itself are foreign objects, and as discussed above, might contribute to the appearance of defects of or damage to the eye. For example, corneal problems like inflammations can occur due to contact lens deposits or decreased oxygen flow as a consequence of wearing a contact lens.

An important and accepted diagnostic tool to assess the physiological state of the cornea is the application of fluorescein to the cornea, followed by the subjective and qualitative interpretation of the observed response.

Although there is debate in scientific literature regarding the value of the diagnostic tool when it comes to light (punctuate) staining, high intensity, gross staining has clearly been associated with corneal lesions, in a number of cases followed by eye-disease. In addition, since contact lens use has become a normal phenomenon in the general population, the number of studies describing corneal staining in contact lens wearers has increased, suggesting a relationship between wearing of contact lenses and staining.

An extensive study by Nichols and others (Optometry and Vision Science, Vol. 79, No. 1, January 2002) showed that at least 55% of the contact lens wearers in the study had some form of corneal staining. Subjects who were noncompliant with their lens care system, or used rewetting drops, or wore conventional lenses without a planned replacement schedule were more likely to have some degree of corneal staining. Non-compliance with care system replacement schedule was associated with moderate-to-severe staining.

It is therefore that under consumers and opticians, corneal staining is considered as a possible indicator of health of the cornea. Therefore, there is need in the field to provide for compositions that can effectively decrease cornea staining when wearing contact lenses. This might be either to alleviate concerns by for example the consumers, or to actually reduce or prevent damage to the cornea, as is diagnosed by the staining described above.

Applicant has therefore performed research and developed compositions that show relatively low, to even no corneal staining when used to soak contact lenses and subsequently applying these contact lenses to the eye of a subject. The compositions described in the current invention show a corneal staining profile that at least meets those profiles of the mainstream of commercial products available, but in many cases, perform even better.

### SUMMARY OF THE INVENTION

It is therefore an object of the current invention to provide for the use of beneficial compounds in compositions that can advantageously be applied to the eye of a subject, in particular persons wearing contact lenses, in order to effectively prevent of even repair damage to the eye/cornea, in particular as a consequence of wearing such contact lens.

More in particular, it is an object of the current invention to provide for the use of compounds in ophthalmic compositions (preferably a solution), in particular contact lens care solutions and/or eye drops, that can be applied to the eye of a subject, in particular persons wearing contact lenses, to prevent or reduce staining of the cornea. Likewise it is an object to provide for compositions which show low cornea staining when applied to the eye, in particular when applied to the eye of a contact lens wearer, even more particular when applied to the eye of a contact lens wearer by treating, soaking, impregnating, dripping, storing, submerging, and the like, a contact lens with/in the composition, in particular a contact lens care solution, according to the invention, and subsequently applying the treated, soaked, impregnated, dripped, stored, submerged, and the like, contact lens to the eye of the contact lens wearer.

### DETAILED DESCRIPTION

It was surprisingly found that the above object can be achieved by the use of compounds, compositions and methods as set-out in the description below and in the claims.

In the following description several terms are used. In order to provide a clear and consistent understanding of the specification and claims, including the scope to be given such terms, definitions are provided. Unless otherwise defined herein, all technical and scientific terms used have the same meaning as commonly understood by one of ordinary skill in the art to which this invention belongs. The disclosures of all publications, patent applications, patents and other references are incorporated herein in their entirety by reference.

In particular, there is provided for the use of carnosine in an ophthalmic composition, in particular a contact lens care solution.

The term “ophthalmic compositions” is understood by the skilled person and relates to a composition intended for use in relationship to the eye. In particular within the context of the current invention, the term “ophthalmic composition” comprises, and preferably consists of, contact lens care solutions and eye drops.

The term “staining” or “corneal staining” or keratitis superficialis punctata refers to abrasions that can show up as bright spots on an otherwise smooth surface of the eye/cornea after the application of fluorescein to the eye, and observed using a light that causes the fluorescein to fluoresce. The method is described in for example Optometry and Vision Science, Vol. 79, No. 1, January 2002. Staining can for example be determined by the following protocol:
Immediately after the removal of the contact lenses, fluorescein sodium was instilled on the superior bulbar conjunctiva using a fluorescein sodium ophthalmic strip (Barnes-Hind Ful-Glo fluorescein sodium ophthalmic strip or equivalent) wetted with a drop of non-preserved buffered saline. Excess fluid was shaken off the strip before instillation, and the fluorescein was instilled on the superior bulbar conjunctiva. Corneal staining can be assessed through a slitlamp biomicroscope with a cobalt blue excitation filter and, optionally a yellow barrier filter (similar to a Wratten filter No. 12) placed over the biomicroscope objective within five minutes of fluorescein instillation. The yellow filter can optionally be used to enhance viewing of fluorescein staining providing a more complete view of epithelial damage.

The term “contact lens care solution (or composition)” relates to an aqueous solution or composition intended to be used for the storage of contact lenses or for treatment of contact lenses, the treatment being for example, caring, cleaning, disinfecting, storing and/or rinsing of contact lenses. Such contact lens care solutions normally comprise compounds directed to the treatment of a contact lens, for example for caring, cleaning, disinfecting, storing and/or rinsing of the contact lens. The term “cleaning” for example means that the solution contains one or more active ingredients in sufficient concentrations to loosen and remove loosely held lens deposits and other contaminants on the surface of the contact lens. The term “disinfecting solution” means a solution containing one or more microbiocidal compounds that is/are effective for reducing or substantially eliminating the presence of microorganisms present on a contact lens. “Storing” relates to keeping contact lenses for a period of time in a contact lens care solution according to the invention, for example overnight, or until the next moment the contact lenses are to be applied to the eye of a contact lens wearer. For example, day lenses can be provided that are stored in a solution without any (usual) preservatives.

Various compounds that normally might be present in such contact lens care solutions are provided herein. Contact lens care solutions can be discriminated from eye-drops as the latter is not directed to the treatment of contact lenses, but intended for direct application of a drop in the eye. In contrast, contact lens care solutions are directed to the treatment of the contact lens outside the eye of the wearer. Obviously, although contact lens care solutions can comprise various compounds providing functionality to the contact lens care solution with respect to the treatment of the eye, such contact lens care solution must be compatible with the eye, in order to not damage the eye, in particular the cornea, when contact lens care solution is transferred to the eye when a contact lens that has been treated with a contact lens care solution is inserted in the eye. Examples of commercially available contact lens care solutions are Eyeye of Barnaux Healthcare in the Netherlands or ReNu of Bausch and Lomb, but within the current invention also included are such solutions used to store contact lenses, for example to store day, week or month contact lenses, before these are provided (for the first time) to a consumer.

As described above, traditionally contact lens care solutions comprise compounds directed to the treatment of the contact lens outside the eye, for example by disinfecting the contact lens or by removing debris from the contact lens, in an attempt to reduce the damage that can be inflicted to the eye, in particular the cornea, as the consequence of wearing the same contact lenses. However, as disclosed herein, it was found that said contact lens care solutions can very effectively be used as a medium for carrying carnosine, which was found to be an important compound for maintaining or improving the health status of the eye, in particular the surface of the eye, in particular the cornea, when a subject is wearing contact lenses.

It was surprisingly found that an ophthalmic composition, preferably an ophthalmic solution, comprising carnosine, preferably said ophthalmic composition further comprising a compound selected from the group consisting of dexpanthenol and hyaluronic acid, even more said composition comprises carnosine, dexpanthenol and hyaluronic acid, provides many beneficial effects to the eyes, in particular to the eyes of a contact lens wearer. These benefits are described in detail below.

In particular it is preferred that the ophthalmic composition is in the form of a contact lens care solution or an eye drop, as will be discussed in detail below. Most preferably, the ophthalmic composition is a contact lens care solution.

It was surprisingly found that when carnosine was included in a ophthalmic composition, preferably a contact lens care solution or composition, contact lenses incubated in such contact lens care solutions or compositions can very advantageously be used to provide carnosine to the eye, thereby preventing the occurrence or manifestation of damage to the eye, in particular the cornea, in particular damage as a consequence of wearing a contact lens. In particular it was found that contact lens care solutions comprising carnosine show low corneal staining, and/or reduce corneal staining in comparison to such contact lens care solution not comprising carnosine.

It is thus with the current invention possible to provide carnosine to the eye/cornea of a subject without that the subject has to take any additional steps (by treating the contact lens as described above, for example by treating the contact lens with a contact lens care solution according to the invention). A person wearing contact lenses normally has to treat, care, clean, rinse, and the like, his contact lenses at least once a day. By now including carnosine in such contact lens care solution, the subject is automatically provided with carnosine after treatment of the contact lens with the contact lens care solution according to the invention upon introduction of a contact lens in the eye, thereby automatically providing carnosine to the eye for preventive or even curative treatment of the eye, in particular the cornea, in a person using, wearing, contact lenses. In particular the current invention allows for the use of carnosine in contact lens care solutions, thereby providing a contact lens care solutions which leads to reduced corneal staining in the eye of a contact lens wearer, when a contact lens is soaked in said contact lens care solution and subsequently the contact lens is applied to the eye, thereby introducing at least part of the contact lens care solution to the eye.

This is surprising as in order for such approach to be successful, the compound included in a contact lens care solution should be taken up by or attach to a contact lens without disturbing the function of the contact lens in the eye (e.g. without providing turbidity or opalescence), should preferably be released from the contact lens in a delayed manner, should preferably be presented to the eye in a form which is immediately available for use by the eye tissue, preferably and in particular by the cornea, preferably, in case of carnosine, without the need of further conversion to an active form and should not be a cause of irritation and should be stable in the contact lens care solution, and should not, for example, interfere (negatively) with a disinfectant when such disinfectant is present in the contact lens care solution.

According to the knowledge of the inventor none of the above has been described or suggested with respect to the compound carnosine, in particular not for use in a contact lens care solutions. In fact, carnosine (beta-alanyl-L-histidine) is a dipeptide of the amino acids beta-alanine and histidine and is also known as (2S)-2-[(3-Amino-1-oxopropyl)amino]-3-(3H-imidazol-4-yl)propanoic acid. As explained above, normally, preferably deposits of proteins and peptides on contact lenses are to be prevented.

Carnosine and some derivatives thereof (carcinine, anserine, balenine, etc.) are known to be among the most important and potent natural (human) (water-soluble) antioxidant agents which act as universal antioxidants both in the lipid phase of cellular and biological membranes and in the aqueous environment protecting lipids and water-soluble molecules especially proteins (including enzymes). Carnosine is also able to avoid carbonylation and glycosylation. This combination of effects of avoiding oxidation, carbonylation and glycosylation is a unique combination. Within the current invention, the term “carnosine” also includes such compounds like carcinine, anserine, and balenine. However preferably only beta-alanyl-L-histidine is used. Carnosine is believed to benefit the re-epithelisation of the cornea that might be required due to damage to said cornea as a consequence of wearing contact lenses.

In other words, by providing for (the use of) carnosine in a contact lens care solution, damage to the eye, in particular the cornea, that might be the consequence of wearing a contact lens can be prevented or even be repaired by wearing the actual contact lens treated with the contact lens care solution. In particular corneal staining was found to be reduced when using carnosine in a contact lens care solution. The current invention thus further improves the comfort of contact lenses and reduces problems as a consequence of wearing such contact lenses, including worries or disorders associated with corneal staining, including, but not limited to lesions, inflammations or infections. In addition, it provides for a highly effective strategy of providing beneficial compounds to the eye, in particular to the eye of a contact lens wearer. A subject wearing contact lenses does not have to add separately eye-drops to its eyes as a preventive measure against or treatment of any damage that might occur as the consequence of wearing contact lenses, or to counteract corneal staining as a consequence of wearing contact lenses in combination with the use of contact lens care solutions not comprising the compounds or combination of compounds according to the current invention. With the current invention the beneficial compounds, including carnosine, will be delivered to the subject's eye automatically when inserting a contact lens in the eye after treatment of the contact lens in a contact lens care solution according to the invention (the treatment for example being the storage of day lenses in an (aqueous) solution comprising at least carnosine before the use of such day lenses by a user; in general such solution preferably does not comprise a preservative or disinfectant).

It is noted that in the art N-acetyl carnosine has been suggested as a compound suitable for the treatment of various eye diseases like cataracts, open-angle primary glaucoma, corneal disorders, and presbyopia (see for example US2004 192647 (A1)). In fact it has been suggested that carnosine per se is unsuitable as a compound in the treatment of such disorders as exogenous carnosine, even when topically administered to the eye, does not accumulate in tissues, but it is excreted with urine or it is preferably destroyed by the enzyme carnosinase, which is present in blood plasma, aqueous humor of the anterior eye chamber, liver and kidney and other tissues, and probably the lens (see for example US2004 192647 (A1)).

WO 9510294 (A1) indeed shows that when 1% N-acetylcarnosine is added to the eye, by such techniques like phonophoresis or subconjunctival injection, carnosine is believed to be made out of N-acetylcarnosine by the eye, which tends to accumulate in the aqueous humour within 15-30 min after the topical administration to the eye. Carnosine administration in itself, by the techniques described above was found ineffective as the carnosine would be quickly metabolised by carnosinase present in the eye.

N-acetylcarnosine has only been described as having a very weak anti-oxidative effect, so application of N-acetyl carnosine in a contact lens care composition does not provide for the same results and efficacy in comparison to the use of carnosine in a contact lens care solution, and according to the current invention.

However, in contrast to what has been described in the prior art, it was now found that when carnosine is comprised in a contact lens care solution, a contact lens treated with such contact lens care solution is a highly effective medium for delivering carnosine directly to the eye, more particular and preferred directly to the cornea, and in particular it is highly effective in lowering/reducing corneal staining. Once such contact lens is inserted in the eye, it can, preferably in a delayed manner (slow-release), release the carnosine comprised in or attached to the contact lens to the eye. In addition it delivers the carnosine directly to those areas of the eye that are in direct contact with the contact lens, and where damage as the consequence of wearing such lenses might most likely occur. This further improves efficacy.

In fact it has now been found that by using carnosine in a contact lens care solution according to the invention, a contact lens incubated in said contact lens care solution and comprising carnosine can, and in contrast to the prior art, provide carnosine to the eye, more in particular to the cornea, of a person wearing contact lenses (in normal day-to-day situations) for a prolonged period of time, preferably for a period of at least 45 minutes, more preferably for a period of at least 60 minutes, even more preferably for a period of at least 2, 3, 4, 5, 6, 7 hours, thereby now allowing to prevent damage to the eye/cornea for prolonged periods of time, in particular for prolonged periods of time preventing damage to the eye, in particular the cornea, in a person wearing contact lenses. In particular it was found that carnosine can advantageously contribute to reduced/lowered staining of the cornea.

According to the current invention, there is no need for the use of N-acetylcarnosine. In a preferred embodiment therefore, N-acetylcarnosine is disclaimed as a compound that can be used according to the current invention.

In fact, while a small number of studies have suggested beneficial effects of N-acetyl carnosine in treating cataracts of the eyes, these and other ophthamological benefits have not been proven. Britain's Royal College of Ophthalmologists assert that neither safety nor efficacy has been sufficiently demonstrated to recommend its use as a topical treatment for cataracts (Revised statement on N-Acetyl Carnosine for Cataracts August 2008; http://www.rcophth.ac.uk/about/publications/). Said statement states that the evidence for the effectiveness of N-acetyl carnosine eye drops is based on experience on a small number of cases carried out by a Russian research team and that to date, the research has not been corroborated and the results have not been replicated by others. Moreover, the long-term effect is unknown. In addition, it states that the evidence base for the safety is in any way insufficient to recommend its use in the short term.

Carnosine is a compound naturally present in the body, not having any safety issues. It is believed that carnosine acts by at least preventing oxidation, inhibit, or even undue lipid peroxidation, preventing oxidative chain reactions, or stabilizing cellular membranes (by providing anti-oxidant activity) in the eye of a subject wearing a contact lens.

In addition to its direct effect to the eye, carnosine (or carnosine and dexpanthenol or carnosine and dexpanthenol and hyaluronic acid (or acceptable salts thereof), or carnosine and hyaluronic acid (or acceptable salts thereof) might provide cleaning or disinfecting properties or reinforce or improve the effect of existing disinfectants optionally present in the contact lens care solution. In other words, carnosine present in a contact lens care solution might act by further improving the cleaning and/or disinfecting properties of such contact lens care solution.

In a preferred embodiment, the carnosine is comprised in the contact lens care solution in a concentration of between 0.01-5% by weight, preferably of between 0.1-2.5% by weight, more preferably of between 0.15-0.5% by weight. It has been found that particular good results can be achieved when the carnosine is present in a contact lens care solution in such amounts, or at such weight percentage.

In another embodiment there is provided for the use of carnosine according to the invention, wherein the contact lens care solution further comprises a compound selected from the group consisting of dexpanthenol, alanine, leucine, isoleucine, valine, and hyaluronic acid, more preferably wherein the contact lens care solution further comprises dexpanthenol.

It has been found that carnosine can advantageously be combined by at least one of the above mentioned compounds. The combination with at least one of the above mentioned compounds provides for a synergistic effect according to the invention by further improving the comfort of wearing a contact lens and further reducing the change of the occurrence of damage to the eye, in particular the cornea, as a consequence of wearing a contact lens, in particular such combination can advantageously be used in contact lens care solution to provide for contact lens care solution that show low, reduced or no corneal staining when a contact lens is treated, for example soaked in such solution and subsequently applied to the eye.

It was found that in particular a combination with dexpanthenol is beneficial. It is believed that the combination of carnosine and dexpanthenol in a contact lens care solution might be advantageous at various levels.

Firstly, it appears that said combination might further improve the functionality of a contact lens care solution with respect to cleaning, disinfecting etc of the contact lens. Consequently, the occurrence of damage to the eye, in particular the cornea, as the direct consequence of wearing a contact lens is reduced when such contact lens was treated with a contact lens care solution comprising carnosine and dexpanthenol in suitable concentrations.

Secondly, due to the (delayed, extended, prolonged, slow-release) delivery of both carnosine and dexpanthenol to the eye by a contact lens that has been treated in a contact lens care solution comprising both carnosine and dexpanthenol, it appears that at least two different mechanisms of preventing damage to cq. early and/or accelerating repair of the eye/cornea as a consequence of wearing a contact lens are effectively used by the eye, and for a prolonged period of time. By the combination of dexpanthenol and carnosine for example, damage to the eye, in particular the cornea, of a subject wearing contact lenses is therefore effectively prevented by improving at least two separate mechanism of preventing or repairing damage to the eye, in particular the cornea, of a subject wearing contact lenses.

Thirdly, and in particular it was found that the combination of dexpanthenol and carnosine is very advantageous in counteracting, lowering/reducing corneal staining. When the combination of these two compounds was included in a contact lens care solution, corneal staining was reduced beyond the level reached by the use of dexpanthenol or carnosine alone, or beyond the level reached by a contact lens care solution comprising neither carnosine nor dexpanthenol. In fact in various subjects, no significant staining of the cornea was reported when using a contact lens care solution comprising carnosine and dexpanthenol. In other words, contact lens wearers using a contact lens care solution comprising the combination of at least carnosine and dexpanthenol to treat, care, soak, rinse, clean, wet, or in any other way treat contact lenses, and subsequently insert such treated lenses in the eye report unexpected reduction in corneal staining, in various cases even absence of any significant or relevant corneal staining.

Dexpanthenol is a stable alcoholic derivate of pantothenic acid, also called Provitamin B5, D-pantothenyl alcohol or D-panthenol. Dexpanthenol can stabilize the lachrymal film and prevent severe losses of the aqueous layer. Dexpanthenol can help against the appearance of dryness, which can lead to a reduced lachrymal film.

Dexpanthenol is used in a preferred contact lens care composition according to the invention in an amount of from about 0.05% to about 10% by weight, preferably in an amount of from about 0.1% to about 4% by weight, more preferably in an amount of from about 0.9% to about 1.8% by weight, based on the total amount of contact lens care compositions which is advantageously formulated in aqueous solution.

In an even further preferred embodiment of the current invention, it was found that a contact lens care solution comprising carnosine, preferably comprising carnosine and dexpanthenol, further comprises hyaluronic acid, and/or sodium hyaluronate and/or other acceptable salts thereof, further beneficial effects as shown herein are provided.

Hyaluronic acid is a linear polysaccharide formed by repeating disaccharide units consisting of D-glucuronic acid and N-acetyl-D-glucosamine linked by beta-1,4 and beta-1,3 glycosidic bonds. Hyaluronic acid is naturally found in many tissues of the body, such as skin, cartilage, and the vitreous humour. The first hyaluronic biomedical product, Healon, was developed in the 1970s and 1980s by Pharmacia, and is approved for use in eye surgery (i.e., corneal transplantation, cataract surgery, glaucoma surgery and surgery to repair retinal detachment). Within the context of the current invention hyaluronic acid also includes any acceptable salt thereof, for example sodium hyaluronate.

In particular it was found that in an ophthalmic composition according to the invention, preferably a contact lens care solution, the combination of dexpanthenol, carnosine and hyaluronic acid is very advantageous in reducing corneal staining. When the combination of these three compounds was included in a contact lens care solution, corneal staining was reduced to or beyond the level reached by the use of dexpanthenol or carnosine alone, the combination of carnosine and dexpanthenol or beyond the level reached by a contact lens care solution comprising neither carnosine nor dexpanthenol. In fact in various subjects, no significant staining of the cornea was reported when using a contact lens care solution comprising carnosine, hyaluronic acid and dexpanthenol. In other words, contact lens wearers using a contact lens care solution comprising the combination of at least carnosine and dexpanthenol, and preferably also hyaluronic acid, to soak, rinse, clean, wet, or in any other way to treat contact lenses, and subsequently insert such treated lenses in the eye report unexpected reduction on corneal staining, in various cases even absence of any significant or relevant corneal staining.

It was found that the use of carnosine, carnosine and dexpanthenol, carnosine and hyaluronic acid, and/or carnosine, hyaluronic acid and dexpanthenol in contact lens care solutions according to the invention was without any significant reduction in the anti-microbial, and/or disinfecting and/or cleaning action of such contact lens care solution. In other words, the above compounds and combinations of compounds can advantageously be used in contact lens care solutions with minimal or little impact on the disinfecting, biocidal, cleaning and/or wetting functions of the contact lens care solution.

Preferably, the hyaluronic acid, or an acceptable salt, for example sodium hyaluronate, is present in the composition according to the invention in an amount of between 0.001 weight % and 0.4 weight %, preferably between 0.005 weight % and 0.2 weight %, even more preferably between 0.005 and 0.1 weight %, even most preferably about 0.01 weight %.

In another embodiment there is provided for the use according to the invention, wherein the contact lens care solution further comprises a disinfectant. Such disinfecting solution according to the invention can be used to disinfect contact lenses against a wide range of microorganisms including, but not limited to Fusarium solani, E coli, Staphylococcus aureus, Pseudomonas aeruginosa, Serratia marcescens, Aspergillus niger and Candida albicans. For contact lenses for example the actual ISO standards 14729 and 14730 need to be met. For the purposes of the present invention the term “disinfect” means the rendering non-viable of substantially all pathogenic microbes that are in the vegetative state, including gram negative and gram positive bacteria, as well as fungi.

It was noted that under circumstances, carnosine present in the contact lens care solution is less stable than expected. It has been found that carnosine might, under circumstances be utilized by bacteria and/or fungi present either in the contact lens care solution (for example introduced upon immersing a contact lens in the contact lens care solution, said contact lens carrying bacteria and/or fungi, for example as were present on the finger top of the subject handling the contact lens, said finger top being used to remove a contact lens from the eye). Next, it was found that by comprising a disinfectant in the contact lens care solution (or by providing sterile contact lens care solution) such undesirable breakdown of carnosine can effectively be reduced.

In another embodiment there is provided for the use according to the invention, and wherein the contact lens care solution further comprises ophthalmically acceptable preservatives, salts, and buffers. Examples thereof are described below and are known to the person skilled in the art.

In another aspect according to the invention there is provided for a pharmaceutical composition comprising carnosine and at least one compound selected from the group consisting of dexpanthenol, hyaluronic acid, alanine, leucine, isoleucine, and valine.

Preferably said pharmaceutical composition is suitable for topical application to the eye, either directly, or by first dissolving or admixing the pharmaceutical composition with a suitable solvent, e.g. sterile water, after which it can be added to the eye. Preferably the pharmaceutical composition comprises a combination of carnosine and a compound selected from the group consisting of dexpanthenol and hyaluronic acid (or acceptable salts thereof), even more preferably the pharmaceutical composition comprises carnosine, dexpanthenol and hyaluronic acid (or acceptable salts thereof). Preferably the composition is suitable for topical application to the eye, even more preferably the composition is present (or in the form of) a contact lens care solution, preferably in addition comprising compounds normally present in contact lens care solutions.

In another aspect there is provided for a contact lens care solution comprising carnosine. In a preferred embodiment, there is provided a contact lens care solution according to the invention further comprising a compound selected from the group consisting of dexpanthenol, alanine, leucine, isoleucine, and valine, more preferably wherein the contact lens care solution further comprises dexpanthenol.

In particular preferred is an ophthalmic composition, preferably a contact lens care solution comprising (% by weight) at least

| | |
|---|---|
| Carnosine | 0.01-5%, preferably 0.1-2.5%, |
| | even more preferably 0.15-0.5%; |
| Dexpanthenol | 0.05-10%, preferably 0.1-4%, |
| | even more preferably 0.9-1.8%; |
| | dissolved in water. |

Optionally, and preferably, further common compounds used in the ophthalmic composition, preferably the contact lens care solutions can be present. For example, the composition may further comprise polyhexanide (polyhexanide biguanide), preferably 0.00001-0.0003% by weight.

In a further preferred embodiment an ophthalmic composition, preferably a contact lens care solution is provided comprising (% by weight)

| | |
|---|---|
| Carnosine | 0.01-5%, preferably 0.1-2.5%, |
| | even more preferably 0.15-0.5%; |
| Dexpanthenol | 0.05-10%, preferably 0.1-4%, |
| | even more preferably 0.9-1.8%; |
| Hyaluronic acid (and salts thereof) | 0.001-0.4%, preferably 0.005/0.2%, |
| | more preferably 0.005-0.1%. |
| Cosmocyl (PHMB) | 0.1-3 ppm (part per million), |
| | preferably about 1 ppm |
| Poloxamer | 0.1-0.4, preferably about 0.25% |
| | dissolved in water. |

Optionally, and preferably, further common compounds used in the ophthalmic composition, preferably contact lens care solutions, can be present. For example, the composition may further comprise polyhexanide (polyhexanide biguanide), preferably 0.00001-0.0003% by weight.

It was found that in particular such compositions can provide for the protection of the eye, in particular the cornea, in particular in a person wearing contact lenses. In particular it has been found that such compositions have a favourable staining profile, i.e. show low/reduced/minimal staining of the cornea when a used as contact lens care solution, i.e. when used to treat a contact lens, after which treatment the contact lens is introduced in the eye of a contact lens wearer.

In accordance with the invention, a contact lens care solution of composition is ophthalmically safe. The term “ophthalmically safe” with respect to a lens care solution is meant that a contact lens treated with the solution is safe for direct placement on the eye without rinsing, that is, the solution is safe and sufficiently comfortable for daily contact with the eye via a contact lens. An ophthalmically safe solution has a tonicity and pH that is compatible with the eye and comprises materials, and amounts thereof, that are believed to be non-cytotoxic.

A contact lens care composition (or solution) according to the invention can be used to treat e.g. clean contact lenses, including hard (PMMA) contact lenses, soft (hydrophilic) contact lenses, gas permeable (RGP) contact lenses, and silicone hydrogels. Preferably the lenses used according to the invention are soft contact lenses. Those soft contact lenses can be hydrogel contact lenses or silicone hydrogel contact lenses. “Hydrogel” refers to a polymeric material that can absorb at least 10 percent by weight of water when it is fully hydrated. Generally, a hydrogel material is obtained by polymerization or copolymerization of at least one hydrophilic monomer in the presence of or in the absence of additional monomers and/or macromers. The contact lenses used according to the current invention can preferably be so-called daily disposable contact lenses (intended to be worn during the day or for a day, after which they are thrown away, and the wearer takes a new set of disposable lenses), or night contact lenses (intended to be worn at night).

In a preferred embodiment, the lens care solution according to the invention is a multipurpose solution capable of disinfecting, cleaning, and rinsing a contact lens.

The list of microbicides which may be employed in the present invention (as a disinfectant (or preservative)) include, but is not limited to, biguanides, biguanide polymers, salts thereof, N-alkyl-2-pyrrolidone, polyquaternium-1, bronopol and hydrogen peroxide. Most preferably, the biguanide is a hexamethylene biguanide polymer (PHMB), also referred to as polyaminopropyl biguanide (PAPB). The PHMB is normally present in the compositions from 0.2 ppm to 5 ppm or from 0.5 ppm to 2 ppm.

Biguanides include the free bases or salts of alexidine, chlorhexidine, hexamethylene biguanides and their polymers, and combinations thereof. One of the more common quaternary ammonium compounds is referred to in the art as polyquaternium-1. Quaternary ammonium compounds are generally referred to in the art as “polyquaternium” disinfectants, and are identified by a particular number following the designation such as polyquaternium-1, polyquaternium-10 or polyquaternium-42. It is to be understood by those skilled in the art that the compositions can include one or more of the antimicrobial components described above.

Typical solutions of this invention contain a microbicide of a PHMB type in an amount of from about 0.01 to about 10 ppm, preferably from about 0.05 to about 5 ppm, more preferably from about 0.1 to about 2 ppm, even more preferably from about 0.2 to about 1.5 ppm.

Where a contact lens care solution or composition comprises a biguanide or a biguanide polymer (e.g., PHMB) as a microbicide, it comprises preferably less than 2500 ppm, more preferably less than 250 ppm, even more preferably no chloride ions.

The present compositions preferably include an effective amount of a chelating component. Any suitable, preferably ophthalmically acceptable, chelating component may be included in the present compositions, although ethylenediaminetetraacetic acid (EDTA), salts thereof and mixtures thereof are particularly effective. Typical amount of EDTA is from about 0.001% to about 1% by weight, based on the total amount of contact lens care composition. However a further advantage of carnosine is that carnosine might act as an effective chelating agent, too, thus allowing to not include a further chelating compound, like EDTA.

The composition of the present invention preferably contains a buffering agent. The buffering agents maintain the pH preferably in the desired range, for example, in a physiologically acceptable range of about 6 to about 8. Any known, physiologically compatible buffering agent can be used. Suitable buffering agents as a constituent of the contact lens care composition according to the invention are known to the person skilled in the art. Examples are boric acid, borates, e.g. sodium borate, citric acid, citrates, bicarbonates, TRIS and phosphate buffers, e.g. Na2HPO4, NaH2PO4, and KH2PO4 or mixtures thereof.

The contact lens care composition according to the invention can preferably comprise a lubricant. “Lubricants” as used herein refer to any compounds or materials which can enhance surface wettability of a contact lens and/or the eye or reduce the frictional character of the contact lens surface. Examples of lubricants include without limitation mucin-like materials and hydrophilic polymers like PVP.

The contact lens care solutions according to the invention are preferably formulated in such a way that they are isotonic with the lachrymal fluid. A solution which is isotonic with the lachrymal fluid is generally understood to be a solution whose concentration corresponds to the concentration of a 0.9% sodium chloride solution. The tonicity of the solution is typically adjusted to be in the range from about 200 to about 450 milliosmol (mOsm).

The inventor has found that when NaCl and/or other charged (dissolved) mineral salts like for example phosphate buffering agents like NaH2PO4 were used to modify the tonicity of a solution comprising carnosine (for example 0.1 to 1 weight % carnosine), the germicidal efficacy of the commonly used agent Cosmocil (for example 1 ppm) can be reduced (even below currently required ISO-14729 standards). It is therefore also provided in the current invention that in an ophthalmic solution comprising carnosine and germicidal (or anti-microbial) agent(s), in particular a charged germicidal anti-microbial agent (when in solution), for example Cosmocil, NaCl and/or other charged dissolved mineral salts like for example phosphate buffering agents like NaH2PO4 are at least partly replaced by another tonicity agent, preferably glycerol or entirely by less charged mineral salts like nitrates, in order to provide for a solution with a tonicity in the range from about 200 to about 450 milliosmol (mOsm), and wherein the same quantity of Cosmocil (in this example 1 ppm) has a better and at least sufficient germicidal efficacy.

Such composition would thus be characterized in that it comprises for example at least a charged anti-microbial agent, for example Cosmocil, for example 1 ppm Cosmocil, at least a non-NaCl tonicity agent, preferably glycerol, and optionally NaCl, to provide for a tonicity (at RT) in the range from 200 to 450 milliosmol, and at least carnosine (for example 0.5 weight %), wherein the solutions in addition meets the ISO-standards with respect to anti-microbial efficacy in ophthalmic solutions, including contact lens care solutions (e.g. for Candida albicans reaching a killing of −log 1 or more (ISO-STANDARD 14729).

The contact lens can be contacted with the solution according to the invention while it is still worn on an eye. The solution according to the invention can be applied directly into an eye to clean and lubricate a contact lens worn on the eye, however preferably the contact lens is in contact with a contact lens care solution according to the invention outside the eye.

In another aspect according to the invention, there is provided for a method for preparing a contact lens comprising carnosine, preferably carnosine and a compound selected from the group consisting of dexpanthenol and hyaluronic acid or even more preferably carnosine and dexpanthenol or carnosine, dexpanthenol and hyaluronic acid, characterized in that the method comprises the steps of incubation a contact lens with carnosine, preferably carnosine and dexpanthenol or carnosine and hyaluronic acid, or even more preferably carnosine, dexpanthenol and hyaluronic acid, preferably the carnosine, preferably carnosine and dexpanthenol or even more preferably carnosine, dexpanthenol and hyaluronic acid, is in the form of a contact lens care solution as defined herein.

It has been found that a contact lens, in combination with a contact lens care solution, can advantageously be used for providing carnosine, preferably carnosine and dexpanthenol or carnosine and hyaluronic acid or even more preferably carnosine, dexpanthenol and hyaluronic acid, to the eye. The contact lens, in particular a soft contact lens, can be used as a vehicle for the prolonged delivery of carnosine, carnosine and hyaluronic acid, preferably carnosine and dexpanthenol or even more preferably carnosine, dexpanthenol and hyaluronic acid, to the eye. It was found that for particular contact lenses, the carnosine, carnosine and hyaluronic acid, preferably carnosine and dexpanthenol or even more preferably carnosine, dexpanthenol and hyaluronic acid, is released in a prolonged (extended) manner from the contact lens to the eye, allowing for prolonged (extended) contact of the eye with the carnosine, carnosine and hyaluronic acid, preferably carnosine and dexpanthenol or even more preferably carnosine, dexpanthenol and hyaluronic acid, in contrast to an initial high level of carnosine, carnosine and hyaluronic acid, preferably carnosine and dexpanthenol or even more preferably carnosine, dexpanthenol and hyaluronic acid, in the eye when an eye-drop would be utilized.

However the use of eye drops comprising carnosine, carnosine and hyaluronic acid, preferably carnosine and dexpanthenol or even more preferably carnosine, dexpanthenol and hyaluronic acid is not excluded. In particular, the use of eye drops comprising carnosine, carnosine and hyaluronic acid, preferably carnosine and dexpanthenol or even more preferably carnosine, dexpanthenol and hyaluronic acid, for decreasing corneal staining, in particular the use of such eye drops by a wearer of contact lenses is within the scope of the current invention. In particular (the use of) such eye drops for reducing corneal staining is provided. In such use, eye drops can be applied directly to the eye, either in the absence or presence of a contact lens.

In case eye drops are provided, these preferably comprise (in weight %, based on the total eye drop composition):
carnosine: from 0.05-5 weight %, preferably 0.1-5 weight %, even more preferably 0.5-4 weight %; and/or
dexpanthenol: from 0.05-10 weight %, preferably 0.1-8 weight %, even more preferably 0.5-2 weight %; and/or
hyaluronic acid in the amounts as described for contact lens care compositions

In another aspect according to the invention there is provided for the use of a contact lens for the administration of carnosine, carnosine and hyaluronic acid, preferably carnosine and dexpanthenol or even more preferably carnosine, dexpanthenol and hyaluronic acid, to the eye of a person wearing a contact lens, wherein the administration comprises, at least once,
a. incubation of a contact lens in a contact lens care solution according to the invention (at least comprising carnosine);
b. introducing said incubated lens in the eye of the person;
c. removing the contact lens from the eye of the person;

In another aspect there is provided for said use of a contact lens wherein the administration further comprises
d. After step c, re-incubating the contact lens in a contact lens care solution according to the invention;
e. re-introducing the contact lens in the eye of the person.

Preferably, said use of the contact lens for administration of carnosine, carnosine and hyaluronic acid, preferably carnosine and dexpanthenol or even more preferably carnosine, dexpanthenol and hyaluronic acid, to the eye comprises that the contact lens is incubated in the contact lens care solution for a period of at least 1 hour, preferably at least 3 hours, more preferably at least 4 hours, most preferably at least 6 hours.

In another preferred embodiment, said use of the contact lens for administration of carnosine, carnosine and hyaluronic acid, preferably carnosine and dexpanthenol or even more preferably carnosine, dexpanthenol and hyaluronic acid, to the eye comprises that the contact lens is present in the eye of the person for a period of at least 1 hours, preferably at least 2 hours, preferably at least 4 hours, and preferably at least 16 hours.

In an embodiment according to the invention, said contact lens care solution can, in addition to carnosine, carnosine and hyaluronic acid, preferably carnosine and dexpanthenol or even more preferably carnosine, dexpanthenol and hyaluronic acid, further advantageously comprise zinc, in particular zinc sulphate, manganese or copper.

In a last embodiment, preferably the use of carnosine, carnosine and hyaluronic acid, preferably carnosine and dexpanthenol or even more preferably carnosine, dexpanthenol and hyaluronic acid, according to the invention, the contact lens care solutions according to the invention, the methods for preparing a contact lens to comprise carnosine, carnosine and hyaluronic acid, preferably carnosine and dexpanthenol or even more preferably carnosine, dexpanthenol and hyaluronic acid, and use of such contact lens in particular relates to a contact lens that is normally used by a subject to provide for improved vision, and not to a contact lens that is not intended to directly correct impaired vision, but that is intended for the treatment of a certain serious eye-disease like cataracts. In other words, the invention is in particular directed to everyday contact lens wearers.

In addition, as has been explained above, the invention in particular relates to subjects wearing contact lenses as an alternative to spectacles (or in combination therewith) for the purpose of improving day-to-day vision. Indeed the use of carnosine, carnosine and hyaluronic acid, preferably carnosine and dexpanthenol or even more preferably carnosine, dexpanthenol and hyaluronic acid, according to the invention is in particular advantageously for subjects wearing lenses for the purpose of improving day-to-day vision.

Although various embodiments according to the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part. Furthermore, titles, headings, or the like are provided to enhance the reader's comprehension of this document, and should not be read as limiting the scope of the present invention. Accordingly, the spirit and scope of the claims should not be limited to the description of the preferred versions contained therein.

Also provided is an ophthalmic composition according to the current invention, preferably a contact lens care solution, comprising carnosine, or carnosine and hyaluronic acid, or preferably carnosine and dexpanthenol, even more preferably comprising carnosine, hyaluronic acid and dexpanthenol, as described herein, wherein the compositions, preferably solution, has a pH value below 7. Preferably such solution comprises nitrates and/or glycerol as described above. It was found that such composition surprisingly does not lead to deforming of particular contact lenses, for example Metafilcon 58 lenses.

### EXAMPLES

The compositions given below are examples of solutions suitable as contact lens care solutions according to the invention. In addition to the compounds mentioned they further may (preferably) comprise such normal and in the field generally known chelating agents like EDTA, pH-buffer systems to maintain a pH of between 6 and 8, where needed, salts or other compounds to provide for a sufficient osmolarity as discussed above and surface active compounds, all in commonly known concentrations.

### Example 1 (% by Weight of Total Solution)

| | |
|---|---|
| Carnosine | 0.1-2% |
| Dexpanthenol | 0.3-4% |
| Polyhexanide | 0.00001-0.0003% (0.1-3 ppm) |

### Example 2 (% by Weight of Total Solution)

| | |
|---|---|
| Carnosine | 0.1-2% |
| Dexpanthenol | 0.3-4% |

### Example 3

To the commercially available contact lens care solution Eyeye, comprising dexpanthenol, of Barnaux Healthcare in the Netherlands, 0.5% by weight carnosine is added.

### Example 4

ReNu MultiPlus Multi-Purpose Solution of Bausch and Lomb is a sterile, isotonic solution that contains hydroxyalkylphosphonate, boric acid, edetate disodium, poloxamine, sodium borate and sodium chloride and is preserved with polyaminopropyl biguanide 0.0001%.

To this solution 0.2% by weight (of the total solution) carnosine is added, to provide solution X.

To this solution X 1.0% by weight (of the total solution) dexpanthenol is added, to provide solution Y,

To this solution Y 0.01% by weight (of the total solution) hyaluronic acid is added to provide solution Z.

### Example 5

Contact lens care solution comprising in water (in percentage by weight, unless indicated otherwise)

| | |
|---|---|
| Dexpanthenol | 1.5% |
| Carnosine | 0.15% |
| Hyaluronic acid | 0.01% |
| Cosmocyl (PHMB) | 1 ppm |
| Poloxamer | 0.5% |
| HPC | 0.1% (hydroxypropylcellulose) |

q.s.: ad pH 7,1 and 275-310 mOsm

### Example 6

The following contact lens care solutions were prepared (all pH 7,1 and 275-310 mOsm), using amounts as defined herein (% by weight of total contact lens care solution), and/or commonly used in the field.

| | A | B | C | D |
|---|---|---|---|---|
| Carnosine | | 0.2 | 0.2 | 0.2 |
| Dexpanthenol | | | 1.5 | 1.5 |
| Hyaluronic | | | | 0.025 |
| acid | | | | |
| Poloxamer | 0.25 | 0.25 | 0.25 | 0.25 |
| HPC | 0.1 | 0.1 | 0.1 | 0.1 |
| PHMB | 1 ppm | 1 ppm | 1 ppm | 1 ppm |
| Glycerol | Ad 290 | Ad 290 | Ad 290 | Ad 290 |
| | mOsm | mOsm | mOsm | mOsm |
| HCl | Ad pH | Ad pH | Ad pH | Ad pH |
| | 7,1 | 7,1 | 7,1 | 7,1 |

The above contact lens care solutions A, B, C or D were provided to subjects and tested for corneal staining. The subjects were allowed to use the contact lens care solution for two weeks, after which corneal staining was determined according to methods known in the art, for example as described in Optometry and Vision Science, Vol. 79, No. 1, January 2002.

Staining was for example determined by the following protocol:
Immediately after the removal of the contact lenses, fluorescein sodium was instilled on the superior bulbar conjunctiva using a fluorescein sodium ophthalmic strip (Barnes-Hind Ful-Glo fluorescein sodium ophthalmic strip or equivalent) wetted with a drop of nonpreserved buffered saline. Excess fluid was shaken off the strip before instillation, and the fluorescein was instilled on the superior bulbar conjunctiva. Corneal staining can be determined through a slitlamp biomicroscope with a cobalt blue excitation filter and, optionally a yellow barrier filter (similar to a Wratten filter No. 12) placed over the biomicroscope objective within five minutes of fluorescein instillation. The yellow filter can optionally be used to enhance viewing of fluorescein staining providing a more complete view of epithelial damage.

Corneal staining was compared to staining as determined prior before the subject was asked to use this contact lens care solution.

Results suggest that in comparison to contact lens care solution A, subjects using B reported slight improvement in corneal staining profile, subjects using C reported strong improvement in the corneal staining profile in comparison to contact lens care solution A, whereas subjects using D reported an even more improved corneal staining profile in comparison to contact lens care solution A, B and C.

Also a combination of carnosine and hyaluronic acid (in the amounts used as mentioned above, and in a contact lens care solution further as described under A above) showed reduction in corneal staining.

### Example 8

Eye drops comprising (in weight %, based on the total eye drop composition):
carnosine: from 0.05-5 weight %, preferably 0.1-5 weight %, even more preferably 0.5 -4 weight %; and/or
dexpanthenol: from 0.05-10 weight %, preferably 0.1-4 weight %, even more preferably 0.5-2 weight %; and/or
hyaluronic acid in the amounts as described for contact lens care compositions.

### Example 9

A contactlens composition or an eye drop comprising (% weight)

| | |
|---|---|
| Carnosine | 0,1-5% |
| Dexpanthenol | 0,1-5% |

A contact lens composition or an eye drop composition comprising (% weight)

| | |
|---|---|
| Carnosine | 0,1-5% |
| Dexpanthenol | 0,1-5% |
| Hyaluronic acid, or an acceptable salt, | 0.001-0.4% |
| for example sodium hyaluronate | |

The above examples were also prepared with a pH value of the solution of below 7,0 in order to obtain a better stability and—for the contact lens solutions—to make certain types of contact lenses compatible with a solution containing carnosine, for example Metafilcon 58 lenses.

1. Ophthalmic composition comprising carnosine preferably said ophthalmic composition further comprises a compound selected from the group consisting of dexpanthenol and hyaluronic acid, even more preferably said composition comprises carnosine, dexpanthenol and hyaluronic acid.

2. Ophthalmic composition according to claim 1, wherein said ophthalmic solution is a contact lens care solution or an eye drop, preferably a contact lens care solution.

3. Ophthalmic composition according to claim 1, wherein said ophthalmic composition is a contact lens care solution, and wherein said contact lens care solution comprises (% by weight)

| | |
|---|---|
| Carnosine | 0.01-5%, preferably 0.1-2.5%, |
| | even more preferably 0.15-0.5%; |
| Dexpanthenol | 0.05 - 10%, preferably 0.1-4%, even more |
| | preferably 0.9-1.8%; and optionally |
| Polyhexanide | 0.00001-0.0003%; dissolved in water. |

4. Contact lens care solution according to claim 2 wherein the composition comprises (% by weight)

| | |
|---|---|
| Carnosine | 0.01-5%, preferably 0.1-2.5%, |
| | even more preferably 0.15-0.5%; |
| Dexpanthenol | 0.05-10%, preferably 0.1-4%, |
| | even more preferably |
| | 0.9-1.8%; and |

Hyaluronic acid (and/or acceptable salts thereof) 0.001-0.4%,
preferably 0.005-0.2%, more preferably 0.005-0.1%.

5. Eye drop according to claim 2 comprising (in weight %, based on the total eye drop composition):
carnosine: 0.05-10 weight %, preferably 0.1-5 weight %, even more preferably 0.5-4 weight %; and
dexpanthenol: 0.05-10 weight %, preferably 0.1-4 weight %, even more preferably 0.5-2 weight %; and, optionally
hyaluronic acid: 0.001-0.4%, preferably 0.005-0.2, even more preferably 0.005-0.1%.

6. Ophthalmic composition, preferably a contact lens care solution, according to claim 1 further comprising a disinfectant, preferably selected from the group consisting of biguanides, biguanide polymers, salts thereof, N-alkyl-2-pyrrolidone, polyquaternium-1, bronopol and hydrogen peroxide, hexamethylene biguanide polymer (PHMB).

7. Ophthalmic composition, preferably a contact lens care solution, according to claim 1 wherein the composition further comprises ophthalmically acceptable surface active ingredient(s), preservatives, germicides, disinfectants, salts and/or buffers.

8. In a method of treating the eye using an eye drop or of maintaining a contact lens using a contact lens care solution, the improvement wherein said eye drop or said contact lens care solution comprises carnosine, or carnosine and hyaluronic acid, preferably carnosine and dexpanthenol, even more preferably carnosine, dexpanthenol and hyaluronic acid.

9. The method according to claim 8 wherein the carnosine is not N-acetyl-carnosine.

10. The method according to claim 8 wherein carnosine is comprised in the contact lens care solution or eye drop in a concentration of between 0.01-5% by weight, preferably of between 0.1-2.5% by weight, more preferably of between 0.15-0.5% by weight.

11. The method according to claim 8 wherein the contact lens care solution is for reducing corneal staining, preferably for reducing corneal staining in the eye of a contact lens wearer.

12. The method according to claim 8, wherein the contact lens care solution further comprises a disinfectant.

13. The method according to claim 8 wherein the solution comprises ophthalmically acceptable surface active ingredient(s), disinfectants, preservatives, salts and buffers.

14. The method of a ophthalmic composition, preferably a contact lens care solution according to claim 8 for reducing corneal staining.

15. The method according to claim 8 wherein the use comprises treating a contact lens, preferably a day contact lens or a night contact lens, with the contact lens care solution.

16. A pharmaceutical composition comprising carnosine, preferably carnosine and a compound selected from the group consisting of dexpanthenol and hyaluronic acid (or acceptable salts thereof), preferably carnosine and dexpanthenol, even preferably carnosine, dexpanthenol and hyaluronic acid wherein the pharmaceutical composition is suitable for administration to the eye, preferably wherein the pharmaceutical composition is in the form of a contact lens care solution or an eye drop.

17. Method for preparing a contact lens comprising carnosine, carnosine and hyaluronic acid, preferably carnosine and dexpanthenol or even more preferably carnosine, dexpanthenol and hyaluronic acid characterized in that the method comprises the steps of incubating a contact lens with carnosine, carnosine and hyaluronic acid, preferably carnosine and dexpanthenol or even more preferably carnosine, dexpanthenol and hyaluronic acid, preferably the carnosine, preferably carnosine and dexpanthenol or even more preferably carnosine, dexpanthenol and hyaluronic acid is provided in the form of a contact lens care solution as defined in claim 1.

18. A method for the administration of carnosine, carnosine and hyaluronic acid, preferably carnosine and dexpanthenol or even more preferably carnosine, dexpanthenol and hyaluronic acid to the eye of a person wearing a contact lens, wherein the administration comprises, at least once,
a) incubation of a contact lens in a contact lens care solution as defined in claim 1;
b) introducing said incubated lens in the eye of the person;
c) removing the contact lens from the eye of the person.

19. The method according to claim 18 wherein the administration further comprises after step c,
d) re-incubating the contact lens in a contact lens care solution as defined in claim 1;
e) re-introducing the contact lens in the eye of the person.

20. The method according to claim 18, wherein the contact lens is incubated in the contact lens care solution for a period of at least 1 hour, preferably at least 3 hours, more preferably at least 4 hours, most preferably at least 6 hours.

21. The method according to claim 18 wherein the contact lens is present in the eye of the person for a period of at least 1 hours, preferably at least 2 hours, preferably at least 4 hours, and preferably at least 16 hours.
